(19) United States
(12) Patent Application Publication
(10) Pub. No.: US 2009/0117195 A1
(43) Pub. Date: May 7, 2009

(54) Hydrophilic Particles Based on Cationic Chitosan Derivatives

(76) Inventors: Peter Kauper, Lausanne (CH); Carsten Laue, Nyon (CH)

Correspondence Address: STURM & FIX LLP, 206 SIXTH AVENUE, SUITE 1213, DES MOINES, IA, 50309-4076, US

(21) Appl. No.: 11/721,764

(22) PCT Filed: Dec. 12, 2005
(86) PCT No.: PCT/IB2005/003733

§ 371 Date: Jun. 14, 2007

(30) Foreign Application Priority Data
Dec. 17, 2004 (IB) IB2004/004269

(51) Int. Cl. A61K 9/14; A61K 47/36; A61K 47/38; A61K 47/30; A61K 38/16; A61K 31/7052; A61P 43/00

(52) U.S. Cl. 424/493; 424/499; 514/777; 514/779; 514/781; 514/772.3; 514/12; 514/44; 977/773; 977/906

(57) ABSTRACT

The present invention provides new nanoparticles and microparticles of hydrophilic character, prepared out of a cationic chitosan derivative and a polyanionic polymer.

### BACKGROUND OF THE INVENTION

1. Field of the Invention

The invention relates to the fields of polymer chemistry, colloid chemistry, polyelectrolyte chemistry, biomedical engineering, pharmaceutical sciences, cosmetic engineering and food industry. More specifically, the present invention relates to a novel nanoparticle system.

2. Description of the Prior Art

Nanosized systems are submicroscopic systems defined by sizes below 1 micrometer. Nanoparticles are submicroscopic colloidal particles. Systems above 1 micrometer in size are named microparticulate. Both, microparticles as well as nanoparticles are used as carrier systems e.g. for drugs, pro drugs, antigens, proteins, vitamins, fragrances, etc. In such systems, microparticles and nanoparticles are formed in a mixture with the molecules of interest to be encapsulated within the particles for subsequent sustained release. Cell encapsulation is a related technology aiming to provide microparticles containing cells.

Many hundreds of combinations of polyanions and polycations were examined for the polyelectrolyte complex formation in hydrophilic microparticulate and nanoparticulate systems. Only a few combinations were found to be usable, and most binary encapsulation systems show instability towards salts and, as a consequence, the polyelectrolyte membrane i.e. the nano- or microparticule disintegrates with time.

To overcome the salt instability of the binary systems, ternary and quaternary systems comprising polyelectrolytes and electrolytes of low molar mass or salts were proposed for microparticulate as well as nanoparticulate systems (S. De and D. Robinson, Polymer relationships during preparation of chitosan-alginate and poly-1-lysine-alginate nanospheres, J. Controlled Release, 89 (2003) 101-112). However, such ternary and quaternary systems imply additional ingredients, involve many preparation steps and hence are complicated to produce.

Only very few binary systems are able to generate usable nanoparticles. The binary system chitosan/sodium tripolyphosphate (TPP) is limited to acidic pH values, due to the insolubility of chitosan at physiological pH (M. J. Alonso Fernandez et al., U.S. Pat. No. 6,649,192). The same is true for the system N-acylated chitosan/TPP (D-W. Lee et al., Physicochemical properties and blood compatibility of acylated chitosan nanoparticles, Carbohydrate Polymers 58(2004)371-377).

Hydrophilic nanoparticles based on polysaccharides, especially based on chitosan, are of growing interest, as witnessed by the growing amount of literature in the field. A recent paper reviews the use of chitosan in micro- and nanoparticles in drug delivery (S. A. Agnihotri, et al., Recent advances on chitosan-based micro- and nanoparticles in drug delivery, Journal of Controlled Release 100 (2004) 5-28). The use of the described particles as delivery means for bioactive molecules such as proteins, peptides, antigens, oligonucleotides, RNA and DNA fragments, growth factors, hormones or other bioactive molecules is nevertheless quite limited, for the following reasons: the preparation of the particles requires physical or chemical interventions which are susceptible to destroy or inactivate such bioactive molecules. One can mention the use of organic solvents, preparation processes involving emulsification, aldehydic crosslinking, acidic preparation conditions, etc.

Chitosan is a natural polymer composed of glucosamine units. It is produced out of crustacean shells or out of biotechnological processes. Chitosan is nearly exclusively derived from chitin by a deacetylation process. Chitosan is available in the market in a variety of forms. Chitosan samples differ in molecular weight and in the degree of deacetylation. Furthermore, chitosan is available in the form of different salts. Chitosan is known for its excellent biocompatibility, and is therefore part of many pharmaceuticals formulations (review article, S. A. Agnihotri, et al., Recent advances on chitosan-based micro- and nanoparticles in drug delivery, Journal of Controlled Release 100 (2004) 5-28), as well as some nanoparticle formulations (e.g. M. J. Alonso Fernandez et al., U.S. Pat. No. 6,649,192, S. De, D. Robinson, Polymer relationships during preparation of chitosan-alginate and poly-1-lysine-alginate nanospheres, J. Controlled Release, 89 (2003) 101-112)). Chitosan is insoluble in aqueous solutions of neutral pH values.

Cationic chitosans are derivatives of chitosan. The chitosan can be modified in different ways to introduce a cationic charge. Modifications, such as alkylation or acylation, can be executed at the amino function of the chitosan to get a quaternized amino group carrying the cationic charge. Or, a moiety can be introduced carrying itself a cationic charge. Said moiety may be covalently linked to one of the chitosan functionalities such as the hydroxyl or the amino group. Of interest are any modifications which result in permanent cationic charges introduced to the chitosan molecule, as they exhibit a pH-independent positive charge. Depending on the amount of cationic charges introduced to the chitosan molecule, water solubility at neutral to basic pH values can be reached, in contrast to unmodified chitosan which is soluble in water only at acidic conditions. A simple way to introduce a permanent cationic charge to the chitosan molecule is the methylation of chitosan's amino groups, resulting in N-trimethyl chitosan salts, notably the chloride form (Sieval et al, Preparation and NMR characterization of highly substituted N-trimethyl chitosan chloride, Carbohydrate Polymers 36(1998)157-165). In vitro and in vivo studies exist describing the bioeffects, particularly the effect as permeation enhancer on mucosal surfaces, of N-trimethyl chitosan chloride (M. Thanou et al., Intestinal absorption of octreoide using trimethyl chitosan chloride: studies in pigs, Pharmaceutical Research, 18(2001)823-828; A. F. Kotze et al, N-Trimethyl chitosan chloride as a potential absorption enhancer across mucosal surfaces: in vitro evaluation in intestinal epithelial cells (Caco-2), Pharmaceutical Research, 14(1997)1197-1202; M. Thanou et al., Intestinal absorption of octreoide: N-trimethyl chitosan chloride (TMC) ameliorates the permeability and absorption properties of the somatostatin analogue in vitro and in vivo, Journal of Pharmaceutical Sciences, 89(2000)951-957).

Despite the important efforts which have been dedicated over the past years to particular systems and chitosan-based micro- and nanoparticles, no such particles have been disclosed which require only two types of polysaccharides and can be easily prepared, without using organic solvents or aldehydic crosslinking, and without requiring strict aqueous conditions (e.g. acidic pH), and without requiring complex equipment such as ultrasound probes or atomizer.

Accordingly, there is a need for such a chitosan-based particular system.

### BRIEF DESCRIPTION OF THE INVENTION

The present invention is directed to hydrophilic particles consisting of one type of cationic chitosan derivative and one type of polyanionic polymer. Said hydrophilic particles may be microparticles or nanoparticles.

In one embodiment of the present invention, said cationic chitosan derivative is a quaternized chitosan derivative, such as N-trimethyl chitosan, N-triethyl chitosan or N-tripropyl chitosan.

In another embodiment of the present invention, said cationic chitosan derivative carries a cationic group covalently linked to the chitosan. For example, said cationic chitosan derivative may be (2-hydroxypropyl trimethyl ammonium) chitosan chloride.

The polyanionic polymer of the present invention may be alginate, carboxymethyl cellulose, sulfoethyl cellulose carboxymethyl amylose or iota carrageenan, but also any other polyanionic polymer.

In a preferred embodiment of the present invention, the cationic chitosan derivative is N-trimethyl chitosan and the polyanionic polymer is alginate.

In a further embodiment of the present invention, the particles have a moiety, a biologically functional group or a prodrug covalently bound to the cationic chitosan derivative, to the polyanionic polymer, or to both.

In still a further embodiment of the present invention, the particles additionally comprise an uncharged polymer, such as polyethylene glycol or a glucan derivative.

The particles according to the present invention may also additionally comprise unmodified chitosan and/or a multivalent cation such as calcium, barium, strontium, aluminium or iron.

The particles according to the present invention may additionally comprise a biologically active substance such as a pharmaceutical, a prodrug, a protein, a nucleic acid, a hormone, a vitamin, a cosmetic, a fragrance or a flavor. Said particles may be used for the transport and the concentration of said biologically active substances in a biological system.

The present invention further provides compositions such as pharmaceutical compositions, cosmetic compositions, food compositions or dermo-pharmaceutical compositions comprising an effective amount of particles according to the invention.

It is also an object of the present invention to provide a process for making hydrophilic particles consisting of one type of cationic chitosan derivative and one type of polyanionic polymer, comprising:
i. preparing an aqueous solution of said polyanionic polymer.
ii. preparing an aqueous solution of said cationic chitosan derivative.
iii. slowly adding the solution obtained in (i) or (ii) to the other solution.

In a preferred embodiment, the process according to the invention is characterized in that the polyanionic polymer is alginate, the cationic chitosan derivative is N-trimethyl chitosan, and the weight ratio between alginate and N-trimethyl chitosan in the respective aqueous solutions is within a range of 1:10 to 1:40.

In a further embodiment, the process according to the invention is characterized in that one or more of the following additional components are present in at least one of the aqueous solutions:
an uncharged polymer selected from the group consisting of polyethylene glycol and glucan derivatives.
a multivalent cation selected from the group consisting of calcium, barium, strontium, aluminium and iron.
unmodified chitosan.

The aqueous solutions of the process according to the invention may also additionally comprise a biologically active substance.

In a further embodiment, the process according to the invention is characterized in that it further comprises steps of incorporating or coating a biologically active substance into or onto the particles after formation of said particles.

In still a further embodiment, the process according to the invention may further comprise steps of incorporating or coating polyanions or polycations into or onto the particles after formation of said particles.

### DESCRIPTION OF THE FIGURES

FIG. 1 is a graph showing the particle size distribution of the nanoparticles obtained in Example 2, measured by a laser diffraction method.

FIG. 2 shows infrared spectroscopy data of nanoparticles from example 1 (from top to bottom: spectrum for nanoparticles from example 1, for N-trimethyl chitosan chloride, and for alginate).

FIG. 3 shows infrared spectroscopy data of nanoparticles from example 4 (from top to bottom: spectrum for nanoparticles from example 4, for nanoparticles from example 1, and for heparin).

FIG. 4 shows infrared spectroscopy data of nanoparticles from example 5 (from top to bottom: spectrum for nanoparticles from example 5, for N-trimethyl chitosan, and for carboxymethyl amylose).

FIG. 5 shows infrared spectroscopy data of nanoparticles from example 8 (from top to bottom: spectrum for nanoparticles from example 8, for N-trimethyl chitosan chloride, and for iota carrageenan).

### DESCRIPTION OF THE INVENTION

In a first embodiment of the present invention, the particles are constituted of only two hydrophilic polymers, one which exhibits a negative charge (polyanion), and a chitosan derivative exhibiting a positive charge (polycation).

Chitosan derivatives permanently charged with cationic charges can be produced following several synthesis routes. One route allows introducing alkyl groups, such as methyl and ethyl, onto the amino group of chitosan backbone (Sieval et al, Preparation and NMR characterization of highly substituted N-trimethyl chitosan chloride, Carbohydrate Polymers 36(1998)157-165). Furthermore, several reaction pathways may be applied to link a moiety, comprising a quaternized amino group, in order to obtain a permanently cationic charged chitosan derivative. (W. Brylak and A. Bartkowiak, New modified cationic oligosaccharides for microcapsule formation, in XII International Workshop on Bioencapsulation, 2004, Vitoria: Universidad del Pais Vasco, Spain, 287-290, J. Y. Kim et al., Synthesis of chitooligosaccharide derivative with quaternary ammonium group and its antimicrobial activity against Streptococcus mutans, Int. J. Biological Macromol. 32 (2003)23-27) Both synthesis strategies can be executed by those trained in the art in a manner that a multitude of different alkyl or different linked moieties are introduced. Those trained in the art may execute the synthesis in manner that the degree of modification, i.e. the amount of total charges introduced into the chitosan backbone, varies (A. Polnok et al., Influence of methylation process on the degree of quaternization of N-trimethyl chitosan chloride, Eur. J. Pharm. Biopharm. 57(2004)77-83).

Different polyanions can be applied in the micro- and/or nanoparticle formation with the cationic chitosan derivatives. Examples are alginate, carboxymethyl cellulose, sulfoethyl cellulose, iota carrageenan and carboxymethyl amylose.

Alginate, the salt of alginic acid is a natural polyanionic polymer composed of mannuronic acid and guluronic acid. It is produced out of algae (mainly brown seaweed) by extraction. Alginate is available in the market in a variety of forms. Alginate samples differ in molecular weight and in composition of mannuronic acid to guluronic acid. Furthermore, alginate is available in the form of different salts, the most common of which is the sodium salt. Alginate is known for its excellent biocompatibility, including when used in nanoparticle formulation (S. De, D. Robinson, Polymer relationships during preparation of chitosan-alginate and poly-1-lysine-alginate nanospheres, J. Controlled Release, 89 (2003) 101-112).

Carboxymethyl cellulose, sulfoethyl cellulose and carboxymethyl amylose are polyanionic polymers obtained by chemical reaction respectively from the natural polymers cellulose and amylose. Depending on the reaction, the carboxymethyl and sulfoethyl group can be introduced in different amounts, in different positions, and/or with different distributions along the chain. The product is available in different degrees of carboxymethylation and sulfoethylation and molar masses, in form of the sodium salt or of other salts.

Carrageenan describes a family of linear polysaccharides derived from red seaweeds; lambda, iota and kappa carrageenan. Due to the presence of sulfate groups they belong to the polyanionic polymer group. They can be distinguished by their different gelling properties: The lambda carrageenan does not gel in water, whereas the iota and the kappa carrageenans form weak and strong gels respectively. Commercially available iota carrageenans differ in molecular composition and molar mass depending on the raw material and the applied extraction methods to obtain the iota carrageenan.

The overall electrical surface charge of these colloidal particles, measurable as zeta potentials, can vary, depending on the ratio of the two hydrophilic polymers. Zeta potentials are found from highly positive values to negative values. After preparation of the nanoparticles, their resulting zeta potential can be adjusted by adding oppositely charged additional ingredients.

The size of the micro- and/or nanoparticles can be modulated as well, from a few nanometers to a few micrometers, by adequately selecting the preparation conditions such as selection of polyanion, concentration of polyanion and polycation, presence and concentration of salts and presence, nature and concentration of uncharged polymers. The size of the micro- and/or nanoparticles can also be selected after the preparation procedure by filtration and/or dialysis techniques.

Another aspect of the present invention is the possibility to covalently link a moiety or functionality to one of the polymer compounds of the micro- and/or nanoparticles prior to particle formation. For applications in the food, cosmetics or pharmaceutical industry or in medicine, such moieties or functionalities might target for example a receptor interaction. Furthermore, a drug or pro drug can be covalently linked to one of the polymer compounds of the particle prior to the micro- and/or nanoparticle formation.

Surprisingly, the formation of the micro- and/or nanoparticles of the present invention occurs spontaneously by a colloid formation of the binary system polyanion and cationic chitosan derivative. The formation of the nanoparticles can be detected by the human eye by the so-called “Tyndall effect”. This term shall refer to light diffusion in many directions by large molecules and small particles resulting in slightly milky solutions. The solvent system for the polyanion as well as for the cationic chitosan derivative can vary from water to salt solutions, and can cover a wide range of pH values, including physiological pH values. To a certain degree, water miscible solvents can be present. This process can also be considered as ionic gelation, ionic crosslinking, coacervation or polyelectrolyte complex formation of the two components. The polyelectrolyte complex formation process is extensively described in literature.

This invention also provides a simple process of nanoparticle preparation, by only dropping one polymeric component in an aqueous solution into another aqueous solution containing the second polymeric compound of opposite charge. No special attention has to be paid to the size of the droplets, or the flow rate of the polymer solution dropped into the second solution. Prior art inventions use techniques in which a nanoscale mist of droplets must be produced, either by a hollow ultrasound probe (A. Prokop, U.S. Pat. No. 6,726,934 and US patent application 20030170313) or by double nozzle atomizer (US patent application 20040136961), or by direct ultrasonication (S. De, D. Robinson, Polymer relationships during preparation of chitosan-alginate and poly-l-lysine-alginate nanospheres, J. Controlled Release, 89 (2003) 101-112).

Nanoparticle formation is affected by the amount (relative proportion) of polyanion dropped into the polycation solution. Depending on the relative proportions of polyanion and polycation, unstable nanoparticles result; for example, when the weight ratio polyanion (alginate) to polycation (N-trimethyl chitosan) is 1:40. In contrast, when the weight ratio 1:20 is reached (as in example 1, below), stable nanoparticles result. Still a lower ratio will lead to a precipitate in the scale of millimeters. It has been observed that particles in the nanoscale (nanoparticles) are efficiently produced whenever the weight ratio of alginate to N-trimethyl chitosan is close to 1:20.

As a result of these proportions, the micro- and/or nanoparticles of the present invention are composed in high excess by the cationic chitosan derivative. Accordingly, the micro- and/or nanoparticle has a high positive zeta potential, due to the pH-independent presence of the positive charges of cationic chitosan derivative, which are not compensated by a counter polyelectrolyte. This is reflected by high positive surface charges of the micro- and/or nanoparticles up to +60 mV (measured with a Zetasizer; MALVERN, UK).

In addition to the polyanion and the cationic chitosan derivative, other components may be added during the micro- and/or nanoparticle formation. Examples are multivalent cations such as calcium, uncharged polymers such as polyethylene glycol, or uncharged glucan derivatives. At acidic pH values, unmodified chitosan can also be present during the micro- and/or nanoparticle formation of a polyanion and a cationic chitosan derivative.

In view of their further use, micro- and nanoparticles according to the present invention may be changed solvents, purified, e.g. by dialysis, sterilized and dried by, wet heat sterilization, freeze drying and spray drying, among other techniques.

The incorporation or coating of charged molecules of interest within or on the micro- or nanoparticles of the present invention (loading) can be achieved by a simple and mild procedure of ionic interaction between the positively charged micro- or nanoparticle, and a negatively or partially negatively charged molecule or an uncharged molecule linked, covalently or by other means, to a moiety carrying negative charges. The incorporation or coating with negatively charged molecules will evidently lower the zeta potential of the resulting micro- or nanoparticle.

The association of bioactive molecules may also comprise mechanisms of physical entrapment. Bioactive molecules of high molar mass or molecules of low molar mass covalently bond to uncharged polymers can be present during micro- or nanoparticle formation, and consequently associated by a physical entrapment process.

The micro- or nanoparticles of this invention are presented as colloidal suspensions in an aqueous medium in which other ingredients could eventually be incorporated, not or partially interacting with the micro- or nanoparticles: organic solvents, salts, acids, bases, cryoprotectives, detergents, preservatives, viscosity enhancers.

One targeted application for the micro- and nanoparticles of the present invention is the delivery and the transport within the human or animal body of bioactive molecules, mainly bioactive macromolecules such as biologically active polysaccharides, proteins, peptides, antigens, oligonucleotides, RNA and DNA fragments, growth factors, hormones etc. Another important targeted application is the delivery within the human or animal body of small organic molecules such as pharmaceuticals. Additional applications include food applications, flavour delivery and fragrance delivery applications.

With respect to the administration routes of micro- and nanoparticles to the human or animal body, the modulation of the zeta potential of the nanoparticles is of importance. Epithelial and mucosal routes, due to the negatively charged surface of the epithelium or mucosa, favor the application of positively charged micro- or nanoparticles. Whereas the parenteral routes, especially intravenous administration, favor the application of neutral to slightly positively or negatively charged micro- or nanoparticles.

The particles of the present invention offer numerous advantages over other types of micro- or nanoparticles previously described in the literature. The main benefits include a simple preparation process, which does not require the use of toxic ingredients such as organic solvents, oils and aldehydic crosslinking agents for incorporating the bioactive molecule of interest in the nanoparticle, does not require strict, specific aqueous conditions (e.g. acidic pH values in the invention disclosed by Alonso Fernandez et al., U.S. Pat. No. 6,649,192). Furthermore, the incorporation of the bioactive molecules of interest into the nanoparticles of the present invention can be carried out with great flexibility under a multitude of conditions, such as different pH values and different salt concentrations. Finally, the physicochemical properties of the micro- or nanoparticles such as their surface charge or their size can be modulated by simple means.

### EXAMPLES

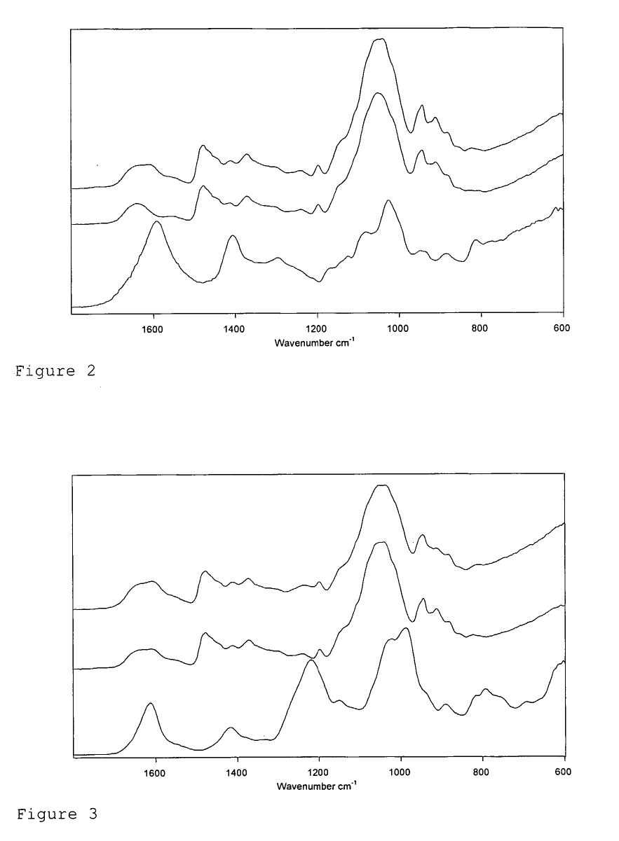

Examples 1 to 9 show the preparation of various types of nanoparticles according to the invention. The chemical compositions of some of these types of nanoparticles are illustrated in FIGS. 2-5, showing infrared spectra. All spectra were recorded on a FTIR spectrometer equipped with an ATR probe (Vector 33, BRUKER, Germany). The spectra are presented in absorbance mode without further processing. Depicted is the so-called fingerprint region from 1800-600 cm⁻¹. After dialysis against water, samples were dried at 60° C. over night and then recorded.

### Example 1

To a solution of 300 ml N-trimethyl chitosan chloride of 0.5% in water at pH7 were added, 100 ml of a solution of 0.1% alginate MediAlg MG5 (middle viscosity type) in water at pH7. Addition was slowly, dropping into a stirred solution. Opalescence appeared already after the first added droplets and became more and more intense. The final dispersion was filtered with a 1.2 μm filter and then dialyzed against water and then concentrated to a final volume of 100 ml by means of a 0.2 μm membrane. A milky, opalescent dispersion with visible Tyndall effect resulted. Dry matter of the final dispersion was determined in an oven at 105° C. to approximately 1%. The nanoparticle diameter was determined to 250 nm, the zeta potential was of approximately +60 mV. Infrared analysis of the dried particles showed a composition of approximately 5:95 alginate to N-trimethyl chitosan. FIG. 2 confirms the presence of alginate beside N-trimethyl chitosan chloride in the nanoparticles. Particularly, the two bands at 1591 cm⁻¹ and 1640 cm⁻¹ of alginate and N-trimethyl chitosan chloride respectively confirm the presence of the two components in the spectrum of the dried nanoparticles.

### Example 2

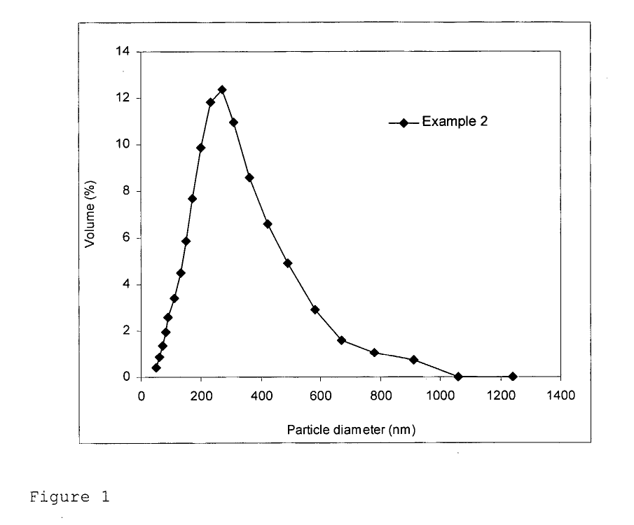

100 ml of a solution of 0.1% alginate MediAlg MG5 in phosphate buffered saline 0.9% NaCl were added to a solution of 300 ml N-trimethyl chitosan chloride of 0.5% in phosphate buffered saline 0.9% NaCl. Addition was slowly, dropping into a stirred solution. Opalescence appeared already after the first added droplets and became more and more intense. The final dispersion was filtered with a 1.2 μm filter, and then dialyzed against water and then concentrated to a final volume of 100 ml by means of a 0.2 μm membrane. A milky, opalescent dispersion with visible Tyndall effect resulted. Dry matter of the final dispersion was determined in an oven at 105° C. to approximately 1%. The nanoparticle diameter was determined to 270 nm. FIG. 1 shows the particle size distribution of the nanoparticles obtained (measurement realized with Mastersizer (MALVERN, UK)). The zeta potential was of approximately +60 mV. Infrared analysis of the dried particles showed a composition of approximately 5:95 alginate to N-trimethyl chitosan.

### Example 3

To a solution of 300 ml alginate of 0.1% in water at pH7 were added, 100 ml of a solution of 0.5% N-trimethyl chitosan chloride in water at pH7. Addition was slowly, dropping into a stirred solution. Opalescence appeared already after the first added droplets and became more and more intense beside a macroscopic flocculation. The final solution was filtered with a 1.2 μm filter and then dialyzed against water and then concentrated to a final volume of 100 ml by means of a 0.2 μm membrane. A milky, opalescent dispersion with visible Tyndall effect resulted. Dry matter of the final dispersion was determined in an oven at 105° C. to approximately 1%. The nanoparticle diameter was determined to 290 nm, the zeta potential was of approximately +20 mV. Infrared analysis of the dried particles showed a composition of approximately 30:70 alginate to N-trimethyl chitosan.

### Example 4

To 50 ml of a dispersion of nanoparticles from example 1 were added, 50 ml of solution of 0.1% heparin in water. The dispersion was filtered with a 1.2 μm filter and dialyzed against water by means of a 0.2 μm membrane. The final dispersion was filtered with a 1.2 μm filter. A milky, opalescent dispersion with visible Tyndall effect resulted which stayed unchanged after filtration through a 0.8 μm filter. FIG. 3 confirms the presence of heparin in the nanoparticles from example 1 by the presence of a band at 1612 cm⁻¹ originated from heparin.

### Example 5

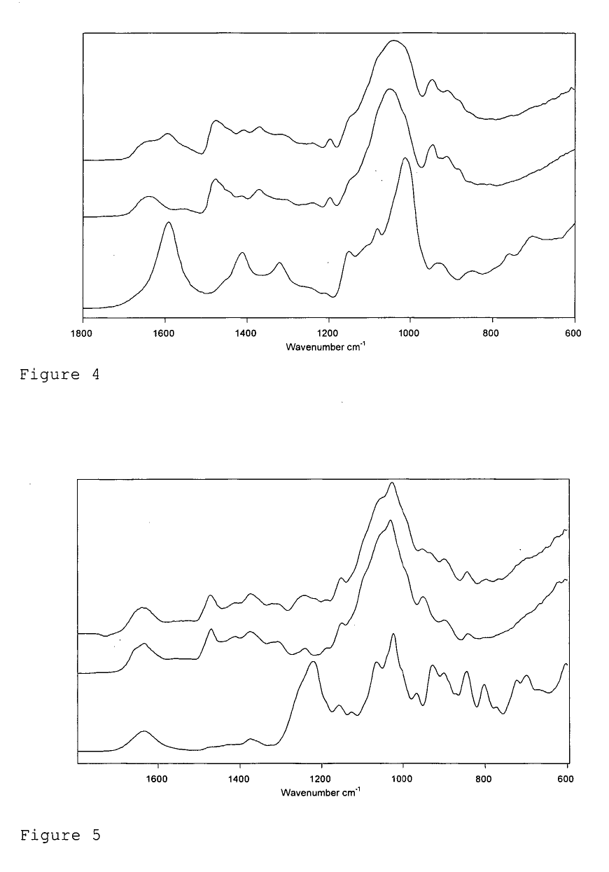

To a solution of 120 ml N-trimethyl chitosan chloride of 0.5% in water at pH7 were added, 80 ml of a solution of 0.1% carboxymethyl amylose in water. Addition was slowly, dropping into a stirred solution. Opalescence appeared already after the first added droplets and became more and more intense. The final dispersion was filtered with a 1.2 μm filter and then dialyzed against water by means of a 0.2 μm membrane. A milky, opalescent dispersion with visible Tyndall effect resulted which stayed unchanged after filtration through a 0.8 μm filter. By filtration through a 0.65 μm filter the intensity of the Tyndall effect was slightly reduced. When using a 0.45 μm filter the intensity of the Tyndall effect was further reduced; and when finally using a 0.22 μm filter, the intensity of the Tyndall effect was again further reduced but still remaining. FIG. 4 confirms the presence of carboxymethyl amylose beside N-trimethyl chitosan chloride in the nanoparticles. Particularly the two bands at 1592 cm⁻¹ and 1640 cm⁻¹ of carboxymethyl amylose and N-trimethyl chitosan chloride respectively present in the spectra of the dried nanoparticles.

### Example 6

To a solution of 60 ml N-trimethyl chitosan chloride of 0.5% in water at pH7 were added, 20 ml of a solution of 0.1% carboxymethyl cellulose in water. Addition was slowly, dropping into a stirred solution. Opalescence appeared already after the first added droplets and became more and more intense. In parallel some macroscopic aggregates became visible. The final dispersion was filtered with a 1.2 μm filter removing the macroscopic aggregates and dialyzed by means of a 0.2 μm membrane against water. A milky, opalescent dispersion with visible Tyndall effect resulted which stayed unchanged after filtration through a 0.8 μm filter. By filtration through a 0.65 μm filter the intensity of the Tyndall effect was reduced. When using a 0.45 μm filter a clear solution was obtained as filtrate.

### Example 7

To a solution of 4 ml N-trimethyl chitosan chloride of 0.5% in water at pH7 were added, 2 ml of a solution of chitosan of pH 4.5 of 0.5%. To this mixture were added, 2 ml of a solution of 0.1% alginate in water at pH7. Addition was slowly, dropping into a stirred solution. Opalescence appeared already after the first added droplets and became more and more intense. The final dispersion was filtered with a 0.8 μm filter. A milky, opalescent dispersion with visible Tyndall effect resulted.

### Example 8

To a solution of 120 ml N-trimethyl chitosan chloride of 0.5% in water at pH5.5 were added, 80 ml of a solution of 0.1% iota carrageenan in water. Addition was slowly, dropping into a stirred solution. Opalescence appeared already after the first added droplets and became more and more intense. The final dispersion was filtered with a 1.2 μm filter and dialyzed by means of a 0.2 μm membrane against water. A milky, opalescent dispersion with visible Tyndall effect resulted which stayed unchanged after filtration through a 0.8 μm and a 0.65 μm filter. By filtration through a 0.45 μm filter the intensity of the Tyndall effect was slightly reduced. When using a 0.2 μm filter the Tyndall effect was significantly reduced. FIG. 5 confirms the presence of iota carrageenan beside N-trimethyl chitosan chloride in the nanoparticles; particularly the two bands at 1241 cm−1 and 1640 cm−1 of iota carrageenan and N-trimethyl chitosan chloride respectively in the spectra of the dried nanoparticles.

### Example 9

To a solution of 40 ml (2-hydroxypropyl trimethyl ammonium chloride) chitosan of 0.5% in water at pH7 were added, 20 ml of a 0.1% solution of sulfoethyl cellulose in water. Addition was slowly, dropping into a stirred solution. Opalescence appeared already after the first added droplets and became more and more intense. Finally, the dispersion was diluted by addition of 120 ml of water. The diluted dispersion was filtered with a 1.2 μm filter and dialyzed by means of 0.2 μm membrane against water. A milky, opalescent dispersion with visible Tyndall effect resulted.

1. Hydrophilic nanoparticles comprising one type of cationic chitosan derivative and one type of polyanionic polymer having at least six negative charges, further wherein their preparation does not require the use of organic solvent.

2. Nanoparticles of claim 1, wherein said cationic chitosan derivative is a quaternized chitosan derivative.

3. Nanoparticles of claim 1, wherein said cationic chitosan derivative carries a cationic group covalently linked to the chitosan.

4. Nanoparticles of claim 2, wherein said quaternized chitosan derivative is selected from the group consisting of N-trimethyl chitosan, N-triethyl chitosan and N-tripropyl chitosan.

5. Nanoparticles of claim 3, wherein said cationic chitosan derivative is (2-hydroxypropyl trimethyl ammonium) chitosan chloride.

6. Nanoparticles of claim 1, wherein said polyanionic polymer is selected from the group consisting of alginate, carboxymethyl cellulose, sulfoethyl cellulose carboxymethyl amylase and iota carrageenan.

7. Nanoparticles of claim 1, wherein said cationic chitosan derivative is N-trimethyl chitosan and said polyanionic polymer is alginate.

8. Nanoparticles of claim 1, wherein a moiety, a biologically functional group or a prodrug is covalently bound to said cationic chitosan derivative, to said polyanionic polymer, or to both.

9. Nanoparticles of claim 1, further comprising an uncharged polymer.

10. Nanoparticles of claim 9, wherein said uncharged polymer is selected from the group consisting of polyethylene glycol and glucan derivatives.

11. Nanoparticles of claim 1, further comprising a multivalent cation selected from the group consisting of calcium, barium, strontium, aluminium and iron.

12. Nanoparticles of claim 1, further comprising unmodified chitosan.

13. Nanoparticles of any claim 1, wherein said nanoparticles further comprise a biologically active substance.

14. Nanoparticles of claim 13, wherein said biologically active substance is selected from the group consisting of pharmaceuticals, prodrugs, proteins, nucleic acids, hormones, vitamins, cosmetics, fragrances and flavors.

15. A composition selected from the group of a pharmaceutical composition, a cosmetic composition, a food composition or a dermo-pharmaceutical composition, comprising an effective amount of nanoparticles as defined in claim 1.

16. Use of the nanoparticles of claim 1 for the transport and the concentration of biologically active substances in a biological system.

17. A process for making hydrophilic nanoparticles consisting of one type of cationic chitosan derivative and one type of polyanionic polymer, comprising:
i. preparing an aqueous solution of said polyanionic polymer.
ii. preparing an aqueous solution of said cationic chitosan derivative.
iii. slowly adding the solution obtained in (i) or (ii) to the other solution.

18. The process of claim 17, wherein said polyanionic polymer is alginate, in that said cationic chitosan derivative is N-trimethyl chitosan, and in that the weight ratio between alginate and N-trimethyl chitosan in the respective solutions is within a range of 1:10 to 1:40.

19. The process of any one of claim 17, wherein one or more of the following additional components are present in at least one of said aqueous solutions:
an uncharged polymer selected from the group consisting of polyethylene glycol and glucan derivatives.
a multivalent cation selected from the group consisting of calcium, barium, strontium, aluminium and iron.
unmodified chitosan.

20. The process of claim 17, wherein a biologically active substance is additionally present in one of said aqueous solutions.

21. The process of claim 17, further comprising steps of incorporating or coating a biologically active substance into or onto said nanoparticles after formation of said nanoparticles.

22. The process of claim 17, further comprising steps of incorporating or coating polyanions or polycations into or onto said nanoparticles after formation of said nanoparticles.
